(19) United States
(12) Patent Application Publication
(10) Pub. No.: US 2024/0382689 A1
(43) Pub. Date: Nov. 21, 2024

(54) TUNING OF AT LEAST ONE THRESHOLD OF A SENSOR OF A DRUG DELIVERY DEVICE OR OF A DRUG DELIVERY ADD-ON DEVICE

(71) Applicant: Sanofi, Paris (FR)

(72) Inventors: Jonathan Jason Drake, Warwick (GB); Anthony Paul Morris, Warwick (GB); Ronald Antony Smith, Salir (PT)

(21) Appl. No.: 18/693,825

(22) PCT Filed: Sep. 22, 2022
(86) PCT No.: PCT/EP2022/076290

§ 371 Date: Mar. 20, 2024

(30) Foreign Application Priority Data
Sep. 24, 2021 (EP) 21315187.1

(51) Int. Cl. A61M 5/315

(52) U.S. Cl. CPC A61M 5/31568; A61M 2205/3306; A61M 2205/3561; A61M 2205/52; A61M 2209/02

(57) ABSTRACT

A method for tuning of at least one threshold of a sensor of a drug delivery device or of a drug delivery add-on device is disclosed. wherein the drug delivery device comprises a movable dosage programming component and the sensor is provided and configured to detect movement of the movable dosage programming component relative to the sensor during dosing of a drug by comparing an output signal of the sensor with at least one threshold, and wherein the method comprises the following steps: calibrating a manufacturing setup rig for sensors of drug delivery devices or drug delivery add-on devices by determining a setting of a threshold for a reference sensor by means of a reference movable dosage programming component and an electronic system configured for processing a signal generated by the reference sensor upon movement of the reference movable dosage programming component and converting the determined setting into a threshold factor; and tuning a threshold of a sensor of a production drug delivery device or of a production drug delivery add-on device by collecting data of the output signal of the sensor by sampling the moving reference movable dosage programming component and calculating at least one threshold based on the collected data using the threshold factor and programming a further electronic system configured for usage with the sensor with the calculated at least one threshold.

### FIELD

The present disclosure relates to the tuning of at least one threshold of a sensor of a drug delivery device or of a drug delivery add-on device.

### BACKGROUND

WO2016131713A1 relates to a data collection device for attachment to an injection device and collecting medicament dosage information therefrom. The data collection device may comprise a mating arrangement configured for attachment to the injection device, a sensor arrangement configured to detect movement of a movable dosage programming component of the injection device relative to the data collection device during delivery of a medicament, and a processor arrangement configured to, based on said detected movement, determine a medicament dosage administered by the injection device. The sensor arrangement may include an optical sensor, for example, an optical encoder unit, particularly including a light source, such as a light emitting diode (LED) and a light detector, such as an optical transducer. The processor arrangement may be configured to monitor a time period elapsed since a pulse was output by the optical encoder and to determine said medicament dosage if said time period exceeds a predetermined threshold.

WO2019101962A1 describes an injection device, which comprises a movable dosage programming component comprising a rotary encoder system having a predefined angular periodicity, and a sensor arrangement comprising a first optical sensor configured to detect movement of the movable dosage programming component relative to the sensor arrangement during dosing of a medicament. The first optical sensor is configured to operate in a strobe-sampling mode at a first frequency. The injection device further comprises a second optical sensor, which is configured to detect movement of the rotary encoder system relative to the second optical sensor and to operate in a strobe-sampling mode at a second frequency lower than the first frequency. Yet further, the injection device comprises a processor arrangement configured to, based on the detected movement of the rotary encoder system, determine a medicament dosage administered by the injection device. The rotary encoder system may be configured to be rotatable with respect to the first optical sensor during a dialing mode of operation of the injection device. The second optical sensor may be configured to operate in a strobe-sampling mode at a second frequency lower than the first frequency. WO2019101962A1 discloses different embodiments of determining a medicament or drug dosage with an optical sensor and a rotary encoder system.

### SUMMARY

This disclosure describes methods and devices for tuning of at least one threshold of a sensor of a drug delivery device or of a drug delivery add-on device.

In one aspect the present disclosure provides a method for tuning of at least one threshold of a sensor of a drug delivery device or of a drug delivery add-on device, wherein the drug delivery device comprises a movable dosage programming component and the sensor is provided and configured to detect movement of the movable dosage programming component relative to the sensor during dosing of a drug by comparing an output signal of the sensor with at least one threshold, and wherein the method comprises the following steps: calibrating a manufacturing setup rig for sensors of drug delivery devices or drug delivery add-on devices by determining a setting of a threshold for a reference sensor by means of a reference movable dosage 15 programming component and an electronic system configured for processing a signal generated by the reference sensor upon movement of the reference movable dosage programming component and converting the determined setting into a threshold factor; and tuning a threshold of a sensor of a production drug delivery device or of a production drug delivery add-on device by collecting data of the output signal of the sensor by sampling the moving reference movable dosage programming component and calculating at least one threshold based on the collected data using the threshold factor and programming a further electronic system configured for usage with the sensor with the calculated at least one threshold. The tuning may be implemented to overcome natural variations inherent in each sensor applied in a drug delivery device or drug delivery add-on device as well as other components, and, thus, improve a correct and consistent operation of each sensor. The natural variations may for example stem from manufacturing tolerances and device characteristics. The calibration step may be performed only once in order to obtain the threshold factor, which is then used in the tuning step, which may be performed with each production drug delivery device or of a production drug delivery add-on device. Thus, the tuning method allows to efficiently tune one or thresholds of production devices instead of calibration each sensor of each single production device. The reference sensor is particularly a sensor different from the at least one sensor of a production drug delivery device or of a production drug delivery add-on device. For example, the reference sensor may be a specifically selected sensor with only small natural variations from the desired sensor specification.

The method is applicable to any drug delivery device having a movable dosage programming component, which is moved relative to a sensor during dosing of a drug. The tuning method may be applied to one or more sensors of a drug delivery device or drug delivery add-on device. For example, in a device with several sensors, it is possible to obtain a threshold factor for each sensor or for a subset of the sensors, and to use the several threshold factors to calculate an overall threshold factor from these, which can then be used to tune the one or more thresholds of production devices.

In an embodiment, the step of calibrating the manufacturing setup rig for sensors of drug delivery devices or drug delivery add-on devices may be iteratively carried out a number times. This may improve the overall calibration result since it can be based on several single calibration results and more precisely represent the general calibration, for example may be an average of the multiple single calibration results.

In embodiments, the step of calibrating the manufacturing setup rig for sensors of drug delivery devices or drug delivery add-on devices may be carried out when at least one parameter of the manufacturing setup rig was changed, which has an influence on the determining of the setting of a threshold. This may be helpful for quality assurance.

In further embodiments, the processing of the signal of the reference sensor may comprise taking a number of readings of the signal at predefined positions of the reference movable dosage programming component with the reference sensor and the electronic system being supplied with at least one predefined electric voltage supply and within at least one predefined ambient temperature range. This may ensure that the calibration is performed under predefined conditions and the results obtained during the calibration step have a high accuracy.

In an embodiment, the processing of the signal of the reference sensor may comprise calculating, for example counting the numbers of taken readings, which are below and which are above a default threshold, and changing the default threshold until the numbers of taken readings, which are below and which are above a default threshold, are nearly equal. This may ensure that the sensor signal may have nearly the same high to low ratio as a target of the movable dosage programming component.

In embodiments, the taken readings of the signal of the reference sensor may be digitized with an analogue-to-digital converter ADC and the threshold factor TF may be calculated as TF=(T−L)/(H−L), wherein T=threshold ADC, L=average of readings <10% maximum, H=average of readings >90% maximum. This allows calculation of the threshold factor in the domain of the ADC from the digitized samples of the reference sensor signal (readings) and the threshold values computed from these readings.

In further embodiments, the sensor may be one of the following: an optical sensing device; an electromagnetic radiation sensing device; a capacitive sensing device; an inductive sensing device; a magnetic sensing device; an ionising radiation sensing device; an acoustic sensing device; an electric current sensing device; an electric voltage sensing device; an acceleration sensing device; a gyroscopic sensing device. Generally, every kind of sensor as applicable with the method, when the sensor is suitable for detecting a movement of the movable dosage programming component and delivers a signal indicative of the movement and comparable to the at least one threshold for determining the movement.

In yet further embodiments, the movable dosage programming component may comprise a rotary encoder system having a predefined angular periodicity and during the calibrating and tuning steps a rotation of the rotary encoder system is detected by the sensor. The rotation may be detected with contact-less sensors such as optical, magnetic, inductive, capacitive sensors. The rotary encoder system may be for example implemented as disclosed in WO2019101962A1 with optical sensors.

In a specific embodiment, the rotary encoder system may comprise 12 nearly equally spaced flags and the sensor takes 300 samples during a 360° rotation of the rotary encoder system, wherein the sampling times are uniformly distributed over the 360° rotation. This has proven to be adequate to allow a threshold for each sensor to be defined with reasonable accuracy. An appropriate interpolation can be used to increase the effective angular resolution, if this would be necessary.

In still further embodiments, the threshold factor may be adjusted for a typical operating voltage of a production drug delivery device or of a production drug delivery add-on device. The typical operating voltage particularly means the operating voltage, for which the production device is designed to be operated. For example, when the threshold factor was determined with a reference operating voltage which differs from the typical operating voltage, it may be adjusted to the typical operating voltage by adjusting it accordingly, particularly by multiplying with a value corresponding to the ratio between the typical and the reference operating voltages.

In a further aspect the present disclosure provides a manufacturing setup rig for sensors of drug delivery devices or drug delivery add-on devices provided for tuning of at least one threshold of a sensor of a drug delivery device or of a drug delivery add-on device, wherein the drug delivery device comprises a movable dosage programming component and the sensor is provided and configured to detect movement of the movable dosage programming component relative to the sensor during dosing of a drug by comparing an output signal of the sensor with at least one threshold, and wherein the manufacturing setup rig comprises: a reference movable dosage programming component; a reference sensor; and an electronic system configured for processing a signal generated by the reference sensor upon movement of the reference movable dosage programming component and converting the determined setting into a threshold factor. This manufacturing rig may be used in the production of drug delivery devices or drug delivery add-on devices to obtain a threshold factor and then to apply this threshold factor when tuning thresholds of production drug delivery devices or production drug delivery add-on devices.

In an embodiment, at least one of the reference movable dosage programming component, the reference sensor and the electronic system may have predefined nominal characteristics being representative of a production drug delivery device or of a production drug delivery add-on device. Thus, it may be ensured that the manufacturing setup rig represents at least in part a production device and the threshold factor was determined with production device like components.

In further embodiments, the manufacturing setup rig may comprise at least one predefined electric voltage supply for the electronic system. This supply may be for example stabilized and deliver a stable electric voltage within a wide operating range so that it may be ensured that the threshold factor can be obtained under predictable conditions.

In yet further embodiments, the manufacturing setup rig may be configured for providing at least one predefined ambient temperature range. Since the ambient temperature may influence the determining of the threshold factor, a predefined ambient temperature range may ensure that also under changing ambient temperatures an accurate result is obtained.

In a yet further aspect the present disclosure provides a production drug delivery device or of a production drug delivery add-on device, wherein the production drug delivery device comprises a movable dosage programming component and the production drug delivery device and/or the production drug delivery add-on device comprise(s) at least one sensor being provided and configured to detect movement of the movable dosage programming component relative to the at least one sensor during dosing of a drug by comparing an output signal of the at least one sensor with at least one threshold, and wherein the at least one threshold of the at least one sensor is tuned with a method as disclosed herein.

### BRIEF DESCRIPTION OF THE FIGURES

FIG. 1 shows an injection device according to an embodiment;

FIG. 2 is an elevated side view of a first embodiment of a rotary encoder system;

FIG. 3 is a plan view of the rotary encoder system shown in FIG. 2;

FIG. 4 is an elevated side view of a second embodiment of a rotary encoder system;

FIG. 5 is a plan view of the rotary encoder system shown in FIG. 4;

FIG. 6 shows a schematic block diagram of an embodiment of a device controller;

FIG. 7 shows a trace of a typical sensor signal with rotation (300 samples per 360° rotation);

FIG. 8 shows an example of typical sequenced readings of a sensor signal with the readings being ordered in sequence based on the magnitude of each reading, so that the lowest reading is the first reading, and the maximum reading is the last reading;

FIG. 9 shows an example of ordered and sequenced readings of a sensor signal with 35th, averaged 40th+60th and 65th percentile reading highlighted, together with ±3 σ variance bar; and

FIG. 10 shows an example of average sensor readings from population of 1000 injection devices with 3 σ deviations limits.

### DETAILED DESCRIPTION OF SOME EMBODIMENTS

In the following, embodiments of the present disclosure will be described with reference to injection devices, particularly an injection device in the form of a pen. The present disclosure is however not limited to such application and may equally well be deployed with other types of drug delivery devices, particularly with another shape than a pen. All absolute values are herein shown by way of example only and should not be construed as limiting.

An example of an injection pen where an injection button and grip are combined and its mechanical construction is described in detail in the international patent application WO2014033195A1. Another example of an injection device where there are separate injection button and grip components is described in WO2004078239A1.

In the following discussion, the terms “distal”, “distally” and “distal end” refer to the end of an injection pen towards which a needle is provided. The terms “proximal”, “proximally” and “proximal end” refer to the opposite end of the injection device towards which an injection button or dosage knob is provided.

First, the construction of an injection pen having a rotary encoder system and the mechanism of dose selection and expelling is explained. This serves to give one in the art a better understanding of the later described tuning of thresholds.

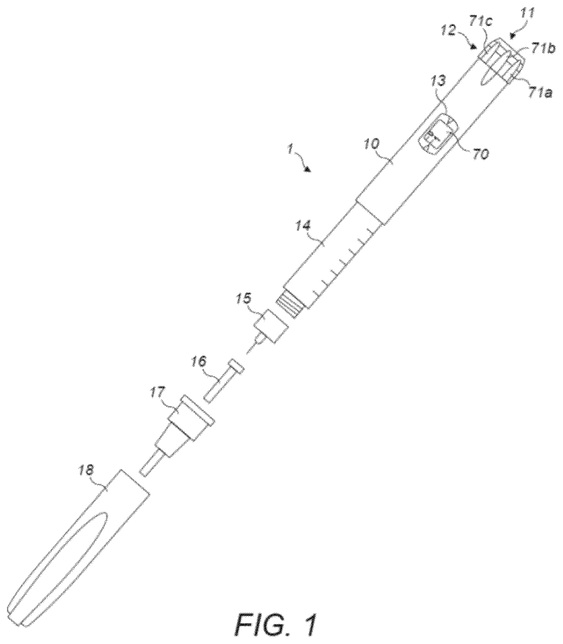

FIG. 1 is an exploded view of an injection pen 1. The injection pen 1 of FIG. 1 is a pre-filled, disposable injection pen that comprises a housing 10 and contains an insulin container 14, to which a needle 15 can be affixed. The needle is protected by an inner needle cap 16 and either an outer needle cap 17 other cap 18. An insulin dose to be ejected from injection pen 1 can be programmed, or ‘dialled in’ by turning a dosage knob 12, and a currently programmed dose is then displayed via dosage window 13, for instance in multiples of units. For example, where the injection pen 1 is configured to administer human insulin, the dosage may be displayed in so-called International Units (IU), wherein one IU is the biological equivalent of about 45.5 micrograms of pure crystalline insulin (1/22 mg). Other units may be employed in injection devices for delivering analogue insulin or other medicaments. It should be noted that the selected dose may equally well be displayed differently than as shown in the dosage window 13 in FIG. 1.

The dosage window 13 may be in the form of an aperture in the housing 10, which permits a user to view a limited portion of a dial sleeve 70 that is configured to move when the dosage knob 12 is turned, to provide a visual indication of a currently programmed dose. The dosage knob 12 is rotated on a helical path with respect to the housing 10 when turned during programming. In this example, the dosage knob 12 includes one or more formations 71a, 71b, 71c to facilitate attachment of a data collection device (drug delivery or injection add-on device).

The injection pen 1 may be configured so that turning the dosage knob 12 causes a mechanical click sound to provide acoustical feedback to a user. The dial sleeve 70 mechanically inter-acts 35 with a piston in insulin container 14. In this embodiment, the dosage knob 12 also acts as an injection button. When needle 15 is stuck into a skin portion of a patient, and then dosage knob 12 is pushed in an axial direction, the insulin dose displayed in display window 13 will be ejected from injection pen 1. When the needle 15 of injection pen 1 remains for a certain time in the skin portion after the dosage knob 12 is pushed, a high percentage of the dose is actually injected into the patient's body. Ejection of the insulin dose may also cause a mechanical click sound, which is however different from the sounds produced when rotating the dosage knob 12 during dialling of the dose.

In this embodiment, during delivery of the insulin dose, the dosage knob 12 is returned to its initial position in an axial movement, without rotation, while the dial sleeve 70 is rotated to return to its initial position, e.g. to display a dose of zero units.

Injection pen 1 may be used for several injection processes until either the insulin container 14 is empty or the expiration date of the medicament in the injection pen 1 (e.g. 28 days after the first use) is reached.

Furthermore, before using injection pen 1 for the first time, it may be necessary to perform a so-called “prime shot” to remove air from insulin container 14 and needle 15, for instance by selecting two units of insulin and pressing dosage knob 12 while holding injection pen 1 with the needle 15 upwards. For simplicity of presentation, in the following, it will be assumed that the ejected amounts substantially correspond to the injected doses, so that, for instance the amount of medicament ejected from the injection pen 1 is equal to the dose received by the user. Nevertheless, differences (e.g. losses) between the ejected amounts and the injected doses may need to be taken into account.

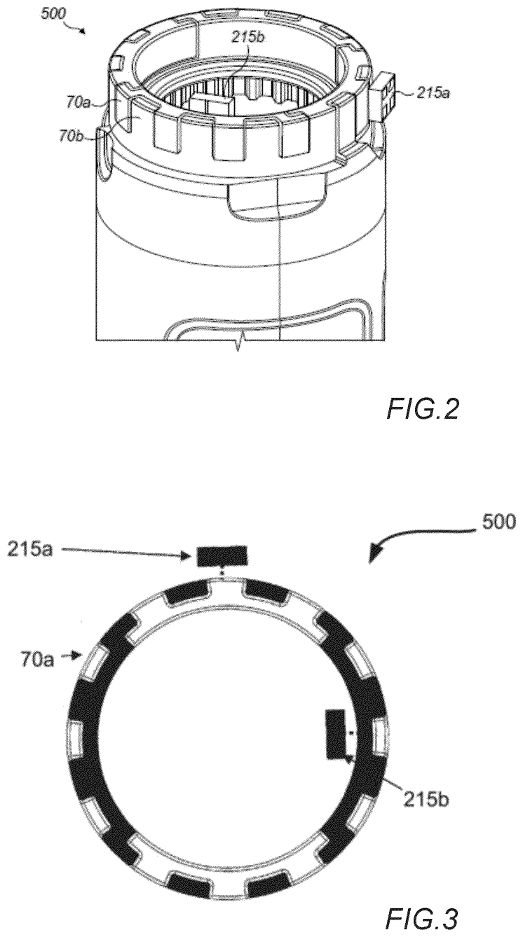

As explained above, the dosage knob 12 also functions as an injection button so that the same component is used for dialling and dispensing. A sensor arrangement 215 (FIGS. 2, 3 and 4, 5) comprising one or more optical sensors may be mounted in the injection button or dosage knob 12 which is configured to sense the relative rotational position of the dial sleeve 70 relative to the injection button 12. This relative rotation can be equated to the size of the dose dispensed or delivered and used for the purpose of generating and storing or displaying dose history information. The sensor arrangement 215 may comprise a primary (optical) sensor 215a and a secondary (optical) sensor 215b. The sensor arrangement 215 may be also mounted in a drug delivery or injection add-on device, which may be provided for usage with different injection devices 1 and configured to collect data acquired with the sensor arrangement 215.

The optical sensors 215a, 215b of the sensor arrangement 215 may be employed with a rotary encoder system, such as the systems 500 and 900 shown in FIGS. 2, 3 and 4, 5, respectively.

The rotary encoder system is configured for use with the device 1 described above and may have a predefined angular periodicity as described in the following.

As shown in FIG. 2 and FIG. 3, the primary sensor 215a and secondary sensor 215b are configured to target specially adapted regions at the proximal end of the dial sleeve 70. In this embodiment, the primary sensor 215a and secondary sensor 215b are infrared (IR) reflective sensors comprising IR light emitting diodes (LEDs) and IR sensitive phototransistors. Therefore, the specially adapted proximal regions of the dial sleeve 70 are divided into a reflective area 70a and a non-reflective (or absorbent) area 70b resulting in the predefined angular periodicity.

The part of the dial sleeve 70 comprising the reflective area 70a and a non-reflective (or absorbent) area 70b may be termed an encoder ring, which has as shown in FIGS. 2 and 3 the predefined angular periodicity defined by the areas 70a, 70b.

To keep production costs to a minimum, it may be favourable to form these areas 70a, 70b from injection moulded polymer. In the case of polymer materials, the absorbency and reflectivity could be controlled with additives, for example carbon black for absorbency and titanium oxide for reflectivity. Alternative implementations are possible whereby the absorbent regions are moulded polymer material and the reflective regions are made from metal (either an additional metal component, or selective metallisation of segments of the polymer dial sleeve 70).

Having two sensors facilitates a power management technique described below. The primary sensor 215a is arranged to target a series of alternating reflective regions 70a and non-reflective regions 70b at a frequency commensurate with the resolution required for the dose history requirements applicable to a particular drug or dosing regime, for example, 1 IU. The secondary sensor 215b is arranged to target a series of alternating reflective regions 70a and non-reflective regions 70b at a reduced frequency compared to the primary sensor 215a. It should be understood that the rotary encoder system 500 could function with only a primary sensor 215a to measure the dispensed dose. The secondary sensor 215b facilitates the power management technique described below.

The two sets of encoded regions 70a, 70b are shown in FIGS. 2 and 3 concentrically with one external and the other internal. However, any suitable arrangement of the two encoded regions 70a, 70b is possible. Whilst the regions 70a, 70b are shown as castellated regions, it should be borne in mind that other shapes and configurations are possible.

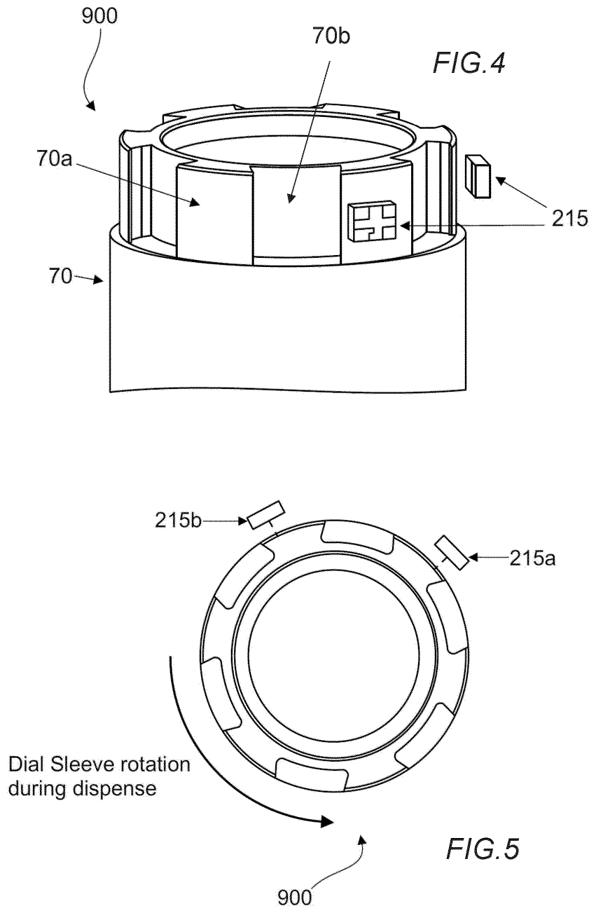

As shown in FIG. 4, the two sensors 215 from this embodiment are configured to target specially adapted regions 70a, 70b of the dial sleeve 70. In this embodiment IR reflective sensors are used, therefore the regions of the dial sleeve 70 are divided into reflective and absorbent segments 70a, 70b. The segments 70a, 70b may also be referred to herein as flags.

Unlike the encoder system 500 described above in relation to FIGS. 2 and 3, the encoder system 900 shown in FIGS. 4 and 5 has both IR sensors 215 target the same type of region 70a, 70b. In other words, the sensors 215 are arranged so that they both face reflective regions 70a or both face absorbent regions 70b at the same time. During the dispensing of a dose, the dial sleeve 70 rotates anti-clockwise 15° relative to the injection button 210 for every medicament unit that has been dispensed. The alternate flag elements are in 30° (or two unit) sections. The sensors 215 are arranged to be out of phase with each other, such that the angle between them equates to an odd number of units (e.g. 15°, 45°, 75°, etc.), as shown in FIG. 5.

The encoder system 900 shown in FIG. 5 has 12 units per revolution, i.e. 12 alternating regions 70a, 70b. In general, embodiments work with any multiple of 4 units per revolution. The angle, or, between sensors 215 can be expressed by the below equation, where both m and n are any integers and there are 4m units dispensed per revolution.

$$α = ( 2 n - 1 ) \frac{360}{4 m} \\ Angle between sensors \qquad Equation$$

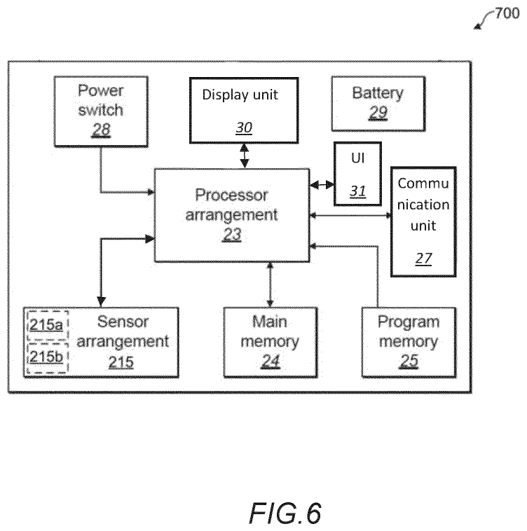

The device 1 or an add-on device for attachment to the device 1 may also include a sensor unit 700, as shown schematically in FIG. 6. The sensor unit 700 may comprise the sensor arrangement 215 including the two sensors 215a, 215b and an electronic system for controlling the sensor arrangement 215 and performing other tasks such as communication with external devices, processing user inputs, outputting information for users etc. The controlling of the sensor arrangement 215 may particularly comprise a driving of at least one of the optical sensors 215a, 215b, wherein driving particularly means how to control an optical sensor to generate light pulses for measurement of a rotation of the encoder ring and to detect reflections of these measurement light pulses from the reflective areas 70a. The electronic system May comprise a processor arrangement 23 including one or more processors, such as a microprocessor, a Digital Signal Processor (DSP), Application Specific Integrated Circuit (ASIC), Field Programmable Gate Array (FPGA) or the like, memory units 24, 25, including program memory 24 and main memory 25, which can store software for execution by the processor arrangement 23, a communication unit or output 27, which may be a wireless communications interface for communicating with another device via a wireless network such as Wi-Fi™ or Bluetooth®, and/or an interface for a wired communications link, such as a socket for receiving a Universal Series Bus (USB), mini-USB or micro-USB connector, a display unit 30, for example a LCD (Liquid Crystal Display), one or more LEDs, and/or an electronic paper display, a user interface (UI) 31, for example one or more buttons and/or touch input devices, a power switch 28, and a battery 29.

The electronic system components 23, 24, 25, 27, 28, 29, 30, 31 may be soldered on a PCB containing the wiring of components. The sensor arrangement 215 may be also attached to the PCB, or may be wired with the processor arrangement 23. The implementation of the sensor unit 700 depends on the drug delivery device or drug delivery add-on device, in which it should be integrated. For example, a PCB with the components 23, 24, 25, 27, 28, 29, 30, 31 may be integrated in the distal end of the injection device 1, and the sensors 215a, 215b may be arranged as shown in FIGS. 2, 3 and 4, 5 and connected to the PCB via wires. At least some of the components 23, 24, 25, 27 may be also comprised by a SoC (System on Chip) or microcontroller.

A firmware stored in the program memory 25 may configure the processor arrangement 23 to control the sensor arrangement 215 such that expelling of a drug dose being delivered with the device 1 can be detected and the sensors 215a, 215b each output a sensor signal corresponding to the detected delivered drug dose, particularly as described above with regard to the FIGS. 2, 3 and 4, 5. The processor arrangement 23 receives the sensor signal of each of the sensors 215a, 215b and takes readings of each sensor signal, which are processed to calculate the delivered dose. A reading may comprise for example one or more voltage samples of an analogue voltage signal of the sensor 215a, 215b. A reading may also comprise an integration of an analogue voltage signal of the sensor 215a, 215b over a certain time span. Instead of voltage signals, also electric currents, electric charges or another output signal generated by a sensor may be used for taking readings, for example frequencies of a sensor signal, frequency shifts. The readings may be taken by each sensor 215a, 215b during operation of the injection device 1 to measure the number of units dispensed by the device 1.

The measuring of the number of dispensed units may comprise counting peaks of each sensor signal and deriving from the counted peaks the delivered dose as described below by in more detail. A peak may be counted when a sensor signal fulfills predefined requirements, particularly exceeding one or more predefined thresholds. For example, when a transition between two encoded regions 70a and 70b is sampled by the optical sensor 215a, 215b, the sensor signal of the sensor 215a, 215b changes its state and, therefore, reflects this transition in its trace as falling or rising of the signal trace. The change of the signal state may be detected by the sensor signal processing performed by the processor arrangement 23 particularly by comparing the sensor signal with one or more predefined thresholds, which may be programmed in an internal non-volatile memory of the processor arrangement 23. The one or more thresholds thus determine when a transition between the regions 70a and 70b is detected. The predefined thresholds may however suffer from natural variations inherent in the sensors as well as other components. In other words, each injection pen differs from each other due to fabrication tolerances and component tolerances, particularly tolerances of the mechanical components of the rotary encoder system and in variations of the characteristics of the electronic system components, particularly the sensors 215a, 215b. Thus, the one or more thresholds of the injection device 1 may be tuned as will be described below by a method comprising a calibration and a tuning step.

It is possible to minimise the power usage of the rotary encoder system 500 so that the size of a battery 29 needed to be packaged into the device 1 can be minimised. The sensors 215a, 215b used in this embodiment require a certain amount of power to operate. This embodiment is arranged such that the sensors 215a, 215b can be switched on and off intermittently at a controlled frequency (i.e. in a strobe-sampling mode). There is inherently a limit to the maximum rotational speed that can be counted by a sampled rotary encoder system before aliasing occurs. Aliasing is the phenomenon where the sampling rate is less than the rate at which sensed regions pass the sensor which means that a miscount could occur when a region change is missed. The secondary sensor 215b with a reduced frequency compared to the primary frequency 215a can tolerate a higher rotational speed before it too becomes aliased. Whilst the secondary sensor 215b is not able to resolve the dose dispensed to the same resolution as the primary sensor 215a, the output of the secondary sensor 215b remains reliable at higher speeds. Therefore both sensors 215a, 215b are used in combination to be able to accurately determine dose delivered up to a first threshold rotational (dispensing) speed. The sensors 215a, 215b can then be used to determine an approximate dose delivered up to a second (higher) threshold dosing speed. At speeds above the second threshold speed the sensors 215a, 215b will not be able to accurately or approximately determine the dose delivered, therefore the second threshold is set above a speed which is not physically possible in the injection pen 1.

The first speed threshold is determined by the sampling rate of primary sensor 215a and the frequency of encoder region transitions, which is fixed at the resolution required by the intended drug or dosing regime (for example one transition per 1 IU). The second speed threshold is determined by the sampling rate of the secondary sensor 215b and the frequency of encoder region transitions. The first threshold is set such that the largest range of dispensing speeds can be covered by the system for accurate reporting of dose dispensed.

The example embodiment shown in FIG. 3 has primary sensor 215a targeting region transitions at 1 transition per 1 IU of dose delivered and the secondary sensor 215b targeting region transitions at 1 transition per 6 IU of dose delivered. Other options are possible which include 1 transition per 2 IU, 1 transition per 4 IU, 1 transition per 8 IU and 1 transition per IU units. These options are each possible because there are 24 separate regions 70a, 70b per revolution in the rotary encoder system 500 shown in FIG. 3. In general, if the number of separate regions 70a, 70b per revolution were n units then there would be options at one region transition per m units where m was any integer factor of n greater than 1 and less than n.

The slower the sampling frequency of both sensors 215a, 215b, the lower the power consumption required and therefore the smaller the required size of the battery 29. It is therefore optimal to minimise, by design, the sampling frequency as far as is practical.

The firmware stored in the program memory 25 and being executed by the processor arrangement 23 for detecting the delivered dose is also implemented to configure the optical sensor 215a and/or 215b to generate measurement light pulses, i.e. to drive the optical sensors 215a, 215b. The optical sensor 215a, 215b can be configured by the processor arrangement 23 to generate light pulses with different sampling rates, particularly with a first sampling rate or with at least one second sampling rate. The first sampling rate is hereby lower than the at least one second sampling rate. The sampling rate determines the time interval of two consecutive light pulses.

Generally, the optical sensors 215a, 215b may have significant tolerances, particularly the phototransistors comprised by the sensors 215a, 215b. Its output is read by an analogue-to-digital (ADC) input on the processor arrangement 23, for example a microcontroller, just as the IR LED of a sensor 215a, 215b is pulsed. Similarly, the tolerances of the IR LED and its drive have some spread. Atop these, the positioning of the sensors 215a, 215b within the housing 10 adds to the tolerances, particularly the distance of the sensors 215a, 215b from the regions 70a, 70b.

In the following, an embodiment of a method for tuning of thresholds of the sensors 215a, 215b is described, particularly the steps taken to characterise the system comprising the sensor arrangement 215 with the sensors 215a, 215b and the rotary encoder system 500, then to transfer that information, as a form of calibration, onto the production devices. Also described are derivations in the use of such a method for device characterisations and evaluations.

1. Overview:
a. Unless otherwise noted within this section, the following description focuses on an injection device that comprises a quadrature incremental rotary encoder utilizing two optical sensors as shown in FIGS. 2 and 3. In this embodiment, both sensors are treated individually but in the same way because they are nominally identical, however, it is not required or important to do so. Nevertheless, it does help simplify the description since the wording is for one sensor but applies to both.
b. There are two steps to this process:
   i. Step 1: Discovering the optimum threshold setting for given reference target and electronic system. This is then converted to a factor (Threshold Factor) that is used to set the production devices. In essence, this step is commissioning of the manufacturing setup rig.
   ii. Step 2: Transferring the calibration to production devices. Each module is passed through the manufacturing setup rig in the same way as the first step and assigned a threshold value for each sensor based upon TF.
c. The two-step process allows faster processing during manufacture because only the second operation is required. Step 1 may be iterative so it could be slow and the data can be thoroughly checked alongside the results. It is only done once, or for QA checks, or after any significant change to the rig, such as changing the encoder ring, etc. Step 2 yields an explicit output for each sensor. Once established it should not require independent checking for each module.
d. Step 1 rotates a reference target (encoder ring) within a standard (representative module that has nominal mechanical and electronic characteristics) taking a number of readings at known rotational positions through the module electronic system. This can be performed at known supply voltage and ambient temperature. From these data, an optimum threshold is iteratively calculated and used to create the Threshold Factor (TF). In essence, this step calibrates the rig.
e. During manufacture, the second step comes into play. The device under test (DUT) or production device is a module that requires setup of the sensors thresholds. In the same manner to Step 1, the reference target is rotated within the DUT whilst collecting the sensors readings. After appropriate parsing of the data, a threshold value is calculated for each sensor of the module using the TF parameter of Step 1. The module is then programmed with those thresholds.

2. Background:
a. In this embodiment, the sensor may be sensitive to supply voltage and temperature. In order to keep the power consumption low and the circuitry size minimal, the sensor stability, with regard these parameters, may be controlled by optimal setup of the signal level threshold at which transitions (for example between the regions 70a and 70b and in case of a Gray code arrangement of the regions 70a, 70b Gray code transitions) takes place. Such can be particularly achieved by an ADC digitisation well in excess of 1 bit, in this case 12 bit although other conversion levels could be used.
b. Due to the digitised nature of the sampled signal, it is possible to apply a setup process to optimise the ADC level representing the threshold for Gray code transitions. This is effectively a calibration using a reference (standard) target to ensure consistency of the electronic system responses.
c. The sensors may be driven in such a manner as to obtain good dynamic range without the signals becoming saturated, e.g. the signal operating range may be linear. In this embodiment, meeting this criterion implies that only just enough power is used for the measurement thereby optimising efficacy.
d. However, if power consumption is not an issue, this process can be used with signals that do saturate and/or those that are non-linear.
e. By setting the ADC to be ratiometric with the supply voltage, partial compensation for variation in sensor signal amplitude with supply voltage is intrinsic to the design. As such, it may be helpful to operate with ADC values, as it is simpler and more stable with changes in supply voltage. This may also be the case when the ADC is set to read absolute and/or difference voltages.

3. Sensor characteristics and threshold setup:
a. The encoder ring (target) may for example have 12 equally spaced flags alternating white and black (e.g. as in FIGS. 2 and 3). A reference target can be rotated exactly 360°, or multiples thereof, within the sensors, taking a sample at a number of accurately defined steps that would typically be uniformly distributed. After suitable processing of the ADC values, each sensor should ideally generate a square wave (implies equal mark-space ratio) when plotted against rotation.
b. As the number of steps increases, the rotational resolution improves, but at some point, a practical limit will be reached whether that be due to the time it takes to complete the measurements or a physical constraint. However, large numbers of steps are not necessary and this method can be the basis of a setup routine that can normalise the encoder electronic system for manufacturing tolerances.
c. The use of such a system to measure and record the readings may typically use 300 samples per sensor across 360°. This may be adequate to allow a threshold for each sensor to be defined with reasonable accuracy. If necessary, appropriate interpolation can be used to increase the effective angular resolution.
d. As flag transitions may be important, this fact can be taken into account during data collection and/or data selection. The data used in this method may start and stop well away from transition regions to ensure no bias in the results.
e. Data may be collected with the device operating at a controlled voltage and known temperature, for example within the range 20° C.±5° C. Using this data (ADC readings), a default threshold can be introduced and the numbers of samples above and below are calculated, for example counted. For each sensor, the threshold level may be changed iteratively, in ADC values, until the numbers of samples above and below are equal. This ensures that each sensor signal, for example used for a Gray code, may have on average, across a full rotation, the same high to low ratio as the target. Other mathematical approaches could be used to find the optimal threshold values.
f. Intrinsic to the thresholds setting is the optimal alignment between the mechanical and sensor transition points. Correct alignment can be assumed when the ratio of the low to high states of the sensor equals that of the black to white ratio of the target.
g. With correct alignment between the mechanical and sensor transition points, it may be then possible to check the phasing between the sensors.
h. In the ADC domain, the target may not yield consistent high level readings (white flags) for many reasons, such as mechanical tolerances, inconsistencies in surface texture, contamination, etc. However, it may be expected within this embodiment that the nominal readings of each flag may be within 10% (other criteria could be used but should be based upon the variance of the reference target) of the maximum across all the flags.
i. The following parameters are defined:
max=global maximum
Hs=Sum values>90% max
Hc=Count readings>90% max
H=average of readings>90% max (equals Hs/Hc)
Ls=Sum values<10% max
Lc=Count readings<10% max
L=average of readings<10% max (equals Ls/Lc)
TF=Threshold Factor
T=Threshold (ADC)
They are related by the following formula:

$$T = L + ( TF * ( H - L ) )$$

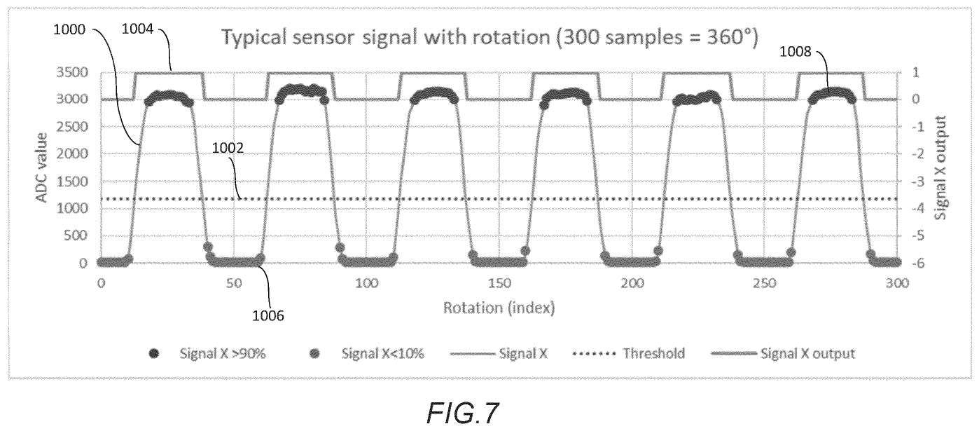

FIG. 7 shows an example trace 1000 of a sensor signal over a 360° rotation of the rotary encoder system with 300 taken samples of the signal 1000. The signal samples 1006 with a value<10% of the minimum sample value (min) and the signal samples 1008 with a value>90% of the maximum sample value (max), the ADC threshold 1002 and the final output signal 1004 generated from comparing the signal samples with the threshold 1002 are also shown in the diagram trace of FIG. 7.
j. The threshold values for the sensors may then be used to calculate a Threshold Factor (TF) for each, via the formula which can be rewritten as TF=(T−L)/(H−L). TF values may then be used to transfer the correct threshold settings to other devices set up through this calibration system.
k. TF can be defined for each sensor or as a single average value covering both. The single value may be typically adequate.
l. If white flag samples and/or black flags are identifiable within a dataset by, for example, indexing then that information can be used to determine the average white flag and average black flag values. Analysis of data from a sample of devices suggests that the threshold may not be strongly dependent upon its method of calculation.
m. With recorded data, it may be possible for other analyses to be done that check for possible fault conditions and/or anomalies. These could include but are not limited to:
   i. White flag levels too low on one sensor or both sensors.
   ii. White flag levels too high on one sensor or both sensors.
   iii. Black flag levels too high on one sensor or both sensors.
   iv. Crosstalk between the sensors.
   v. Incorrect phasing.
   vi. Digitisation faults: Incorrect digitisation level, stuck bit/s, multiplexing faults, bit/s inversion, missing levels, etc.
   vii. Excessive noise.
   viii. Drift in the sensor/s signal/s.
   ix. Spurious readings.
n. The threshold factor can typically be adjusted to be appropriate for the typical operating voltage of a battery power supply of the injection pen, e.g. a coin cell, for example 2.5 V or 2.7 V. Repeating the data collection process at various supply voltages and/or temperatures may provide data that can facilitate adaptive adjustments of the thresholds, signal levels, or both, that can be done mathematically within the microcontroller and/or another computer to compensate for metrics for which the single calibration process could be insufficient or inappropriate. These adjustments can be implemented in many ways, for example but not limited to, look-up tables, continuous function models, step functions, etc. They could be done in near real time or as part of subsequent processing.
o. Implicit in this methodology may be the following:
   i. The reference electronic system and reference target used for the system setup may be representative of those used in the pen devices.
   ii. There may be a consistency between the manufactured parts and test rig.
   iii. Systemic and device variances may be linear or approximate to linear over the range of interest.
   iv. Responses to temperature and voltage may be consistent across devices.
p. Although this process was designed around the embodiment of an injection pen that comprises a quadrature incremental rotary encoder utilizing two optical sensors as shown in FIGS. 2 and 3, it is applicable to other encoder devices in which the signal from a sensor is digitised to form a digital representation. It is not limited to rotary encoder systems. Such systems/devices may comprise one or more sensors using, but not limited to, electromagnetic radiation sensing, capacitive sensing, inductive sensing, magnetic sensing, ionising radiation sensing, acoustic sensing, current sensing, voltage sensing, acceleration sensing, gyroscopic sensing, etc.
q. The process may be applicable to encoder systems with one or more sensors.
r. Other system characterisations, investigations and evaluations may be performed using this methodology or a derivation thereof. These may include but are not restricted to:
   i. Effects from external illumination.
   ii. Effects from acoustic noise, shocks and vibrations.
   iii. Fluid ingress influences.
   iv. Checking EMC (Electromagnetic Compatibility) compliances.
   v. Atmospheric conditions such as humidity, pressure, etc.
   vi. Ionising radiations effects.

In a further embodiment, sensor readings may be captured in a similar way to the method described above, such that 300 readings are acquired for each sensor at equal rotational spacing within a full 360° rotation of the encoder target.

To capture readings during manufacture of each device, it may be possible to communicate remotely with the device, via a wireless communication protocol, which may be the same wireless protocol provided on the device whilst in use. Alternatively, readings may be captured via an alternative wireless communication protocol, or via a physical connection to the device, which may not be available once assembly of the device has been completed. For example, the communication unit 27 of the sensor unit 700 may comprise a wireless communication module via which a remote communication with an external device may be conducted in order to capture the readings. The external device may be for example a computing device configured to perform computations as described above for determining a threshold setting and converting the setting to a threshold factor.

If a physical connection is made to the device, it may be possible to provide external power to the device. The readings may therefore be captured at a known and consistent supply voltage, which may be different from the typical operational voltage of the device. It may be beneficial to capture readings at a higher voltage, so as not to draw energy from the embedded power source of the device under test. It may therefore be beneficial to capture readings at or around the nominal open circuit voltage of a single cell, such as a lithium coin cell, at a voltage around 3.1V, rather than at the typical operational voltage of the device, powered solely from a lithium coin cell, which may provide an operational voltage of between 2.9V to 2.3V dependent on the age, usage and temperature of the device, once under load.

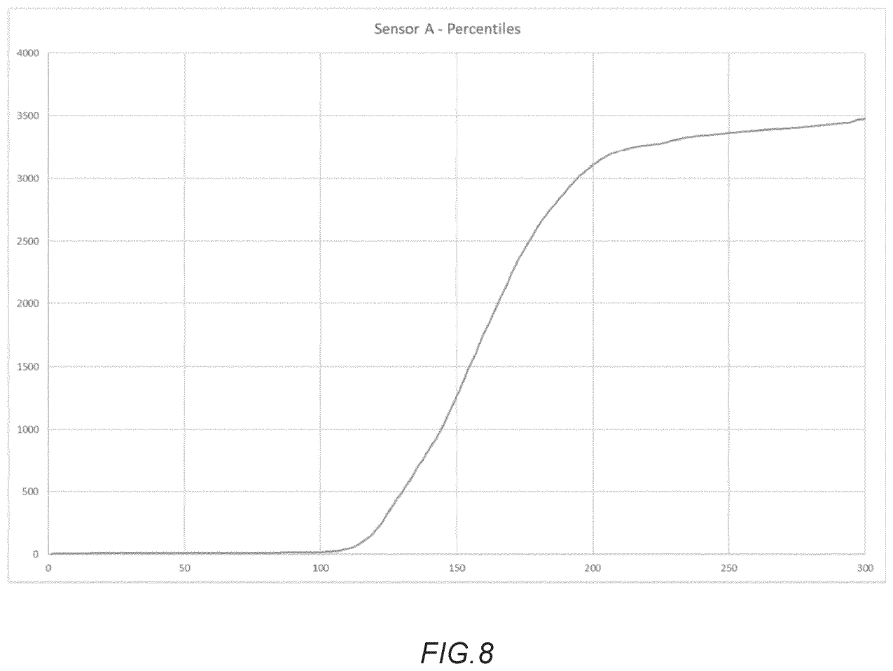

Once the sensor readings have been captured, the 300 readings can be ordered in sequence based on the magnitude of each reading, so that the lowest reading is the first reading, and the maximum reading is the last reading. An example of readings ordered in such a sequence is shown in FIG. 8. Applying this method the 150th reading is the median or 50th percentile reading, with 150 reading being less than or equal to the median reading, and 150 readings being greater than the median reading. As the readings were captured at equal rotational spacing within a full 360° rotation, this implies that the sensor readings were above the median reading for 180° of the 360° rotation.

If the encoder target comprises 12 equally spaced alternating white and black flags, so that each white flag covers 30° and each black flag covers 30°, and the response of each flag is considered symmetrical about its own centre, these ordered readings can be considered to represent the average sensor readings during a 30° rotation of the encoder target, from the centre of a black flag to the centre of a white flag.

Furthermore, if the device contains two sensors positioned around the same encoder target, to provide a quadrature Gray code sequence, similar to the embodiment shown in FIGS. 2 and 3, there will be 24 transition of a 2 bit Gray code during a single 360° rotation. If the sensors are positioned to provide equal spacing of the Gray code transitions, a transition would nominally occur every 15° of rotation of the encoder system.

It may be desirable, when the device is at rest, for the encoder system to provide a robustly determined encoder state (i.e. to be as far away from a transition as possible). Therefore, it may be preferred for the nominal design to be 7.5° from a Gray code transition in any 24 possible rest states of the device (rest state is achieved when the device is in dialling mode).

Mechanical tolerances across numerous components within the full device will create an overall rotational tolerance across the encoder system, which may cause any given device within the population of manufactured devices to be closer to a transition when in the rest state, than the 7.5° nominal design.

For example, the combined rotational tolerance may be in ±3°. Any given device may therefore be within 4.5° of a transition when in the rest state.

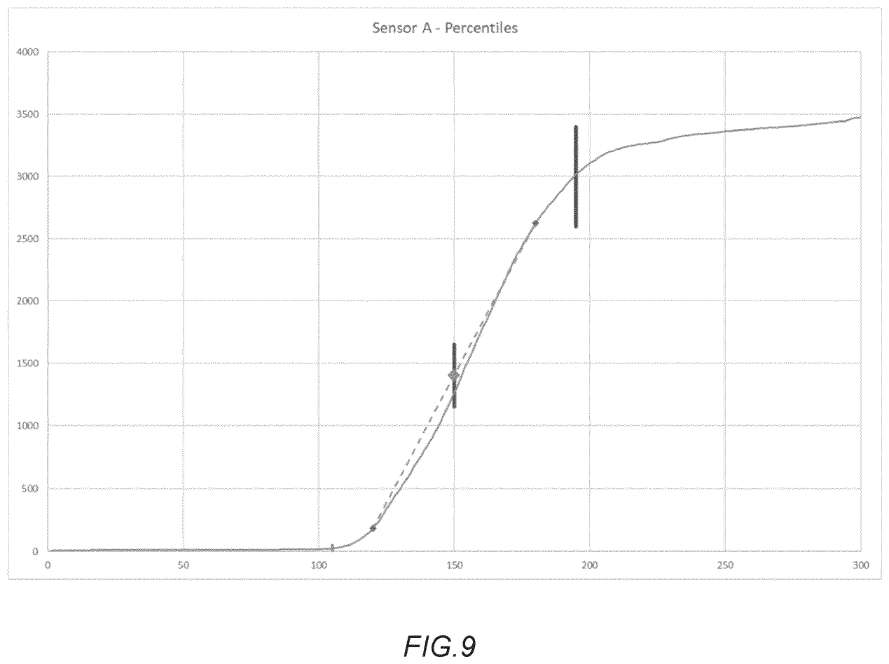

Given this example, considering the 300 sensor readings in magnitude order, captured at equal rotational intervals, it can be inferred that the 35th percentile and the 65th percentile readings occur are 4.5° rotation from the median value (being ±15% away from the median value, with an overall range of 30°, hence ±4.5° away from the median). Hence it may be desirable, for robust digitisation of the sensor reading when in the rest states, for there to be a large margin between the 35th and 65th percentile readings and the threshold at which the sensor reading is digitised to be 0 (black) or 1 (white). An example of ordered and sequenced readings with the 35th, averaged 40th+60th and 65th percentile reading highlighted, together with ±3 σ variance bar is shown in FIG. 9.

In this embodiment, the threshold value can be determined by considering the median or 50th percentile reading. The threshold value can also be determined by considering the average of two or more readings spaced equally either side of the 50th percentile. For example, the threshold value may be determined by considering the average of the 40th & 60th percentile value. Utilising the average of multiple percentile readings either side of the 50th percentile may reduce the coefficient of variation across the population of production devices.

If the sensor readings are recorded at a supply voltage other than the expected operational voltages of the device, a scale factor may be applied to the threshold, to compensate for the effect of voltage variation on sensor reading. This voltage compensation factor can be determined through development tests, capturing sensor readings from a single device and encoder target at a range of supply voltages. Particularly, a linear compensation factor can be applied, or that a more complex factor can be applied based on empirical data.

Based on an indication of further empirical data, an additional compensation factor may be applied, which may be based on observed differences in sensor reading between the manufacturing test rig and a population of fully assembly devices.

The following equation is an example of a threshold calculation for the above described method:

$$Threshold = ( 40 th percentile + 60 th percentile ) / 2 * Operational Voltage / Supply Voltage$$

Operational voltage may be 2.3V
Supply voltage may be 3.1V

During the life of each device, sensor readings may be captured to determine the delivered doses at a range of operational voltages. These voltages may range from 3.1V on first use, during manufacturing tests, to a typical operational voltage of 2.7V to 2.5V during normal usages, and a voltage around 2.3V towards the end of life of the device. The injection device may be configured to warn the user, for example via the display unit 30 of the sensor unit 700 (FIG. 6), when then voltage has reduced to <2.5V, and to create a further end-of-life warning then the voltage has reduced to <2.3V. If the injection device is stored in a cold environment, low voltages may occur early in the life of the device.

Device thresholds may therefore be also calculated based on a lower range of operational voltage, which may result in a reduction in the sensor reading for a given configuration of the encoder system. At lower operational voltage, sensor readings when facing a white target may be significantly lower, whilst sensor readings when facing a black target may be minimally changed.

During manufacturing tests, once the device with the encoder system and dispensing mechanism is fully assembled, further quality assurance tests can be conducted. These tests may capture sensor readings at the highest operational voltages, as the battery, particularly a coin cell would be new and unused, and the temperature of the manufacturing environment may be controlled. If thresholds are set based on a lower operational voltage, a manufacturing test may confirm that the injection device function is robust whilst operating at a higher voltage.

Conversely, if the thresholds are set based on nominal or high operational voltages, the manufacturing test may not be able to confirm that device function is robust whilst operating at lower voltages.

Sensor reading capture and threshold calculation may be performed independently for each sensor in the encoder system.

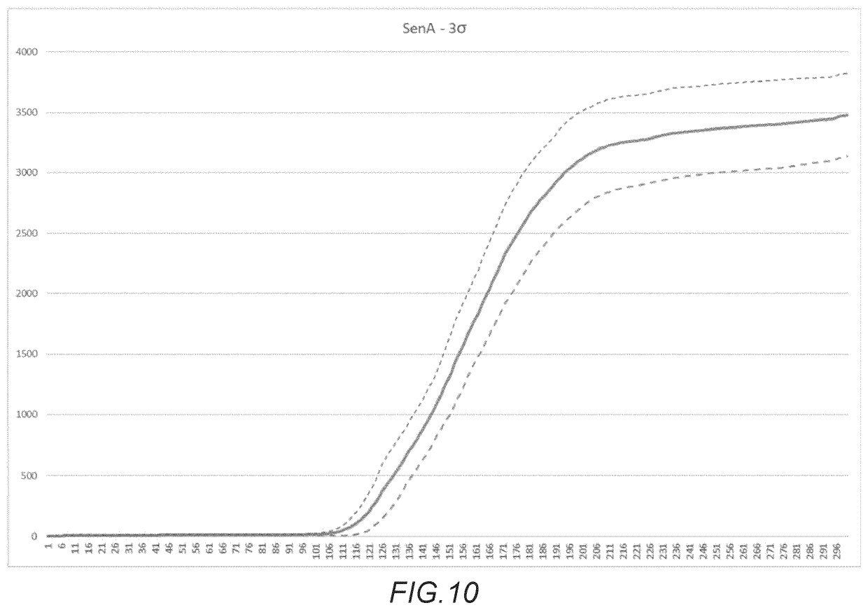

Acceptance limits can be applied to any kth percentile value, or range of percentile values, to achieve statistical process control and ensure functional acceptance limits are not exceeded. Development of functional acceptance limits may be based on empirical data captured during development build phases. Statistical process control limits may be based on continuous monitoring of the manufacturing process and sensor reading data, to determine outliers within a normally disrupted population of devices. FIG. 10 shows an example of average sensor readings from population of 1000 injection devices with 3 σ deviations limits.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber May be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly (A21), Arg (B31), Arg (B32) human insulin (insulin glargine); Lys (B3), Glu (B29) human insulin (insulin glulisine); Lys (B28), Pro (B29) human insulin (insulin lispro); Asp (B28) human insulin (insulin aspart); human insulin, wherein proline in position B28 is replaced by Asp, Lys, Leu, Val or Ala and wherein in position B29 Lys may be replaced by Pro; Ala (B26) human insulin; Des (B28-B30) human insulin; Des (B27) human insulin and Des (B30) human insulin.

Examples of insulin derivatives are, for example, B29-N-myristoyl-des (B30) human insulin, Lys (B29) (N-tetradecanoyl)-des (B30) human insulin (insulin detemir, Levemir®); B29-N-palmitoyl-des (B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des (B30) human insulin, B29-N-omega-carboxypentadecanoyl-gamma-L-glutamyl-des (B30) human insulin (insulin degludec, Tresiba®); B29-N-(N-lithocholyl-gamma-glutamyl)-des (B30) human insulin; B29-N-(ω-carboxyheptadecanoyl)-des (B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′) 2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′) 2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1: 2014 (E). As described in ISO 11608-1: 2014 (E), needle-based injection systems may be broadly distinguished into multi-dose container systems and single-dose (with partial or full evacuation) container systems. The container may be a replaceable container or an integrated non-replaceable container.

As further described in ISO 11608-1: 2014 (E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1: 2014 (E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1: 2014 (E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

1. A method for tuning of at least one threshold of a sensor (215; 215a, 215b) of a drug delivery device (1) or of a drug delivery add-on device, wherein the drug delivery device (1) comprises a movable dosage programming component (70, 70a, 70b) and the sensor (215; 215a, 215b) is provided and configured to detect movement of the movable dosage programming component (70, 70a, 70b) relative to the sensor (215; 215a, 215b) during dosing of a drug by comparing a output signal (1004) of the sensor (215; 215a, 10) 215b) with at least one threshold, and wherein the method comprises the following steps:
calibrating a manufacturing setup rig for sensors (215; 215a, 215b) of drug delivery devices (1) or drug delivery add-on devices by determining a setting of a threshold for a reference sensor by means of a reference movable dosage programming component and an electronic system configured for processing a signal generated by the reference sensor upon movement of the reference movable dosage programming component and converting the determined setting into a threshold factor; and
tuning a threshold of a sensor (215; 215a, 215b) of a production drug delivery device (1) or of a production drug delivery add-on device by collecting data of the output signal (1004) of the sensor (215; 215a, 215b) by sampling the moving reference movable dosage programming component and calculating at least one threshold based on the collected data using the threshold factor and programming a further electronic system configured for usage with the sensor with the calculated at least one threshold.

2. The method of claim 1, wherein the step of calibrating the manufacturing setup rig for sensors (215; 215a, 215b) of drug delivery devices (1) or drug delivery add-on devices is iteratively carried out a number times.

3. The method of claim 1 or 2, wherein the step of calibrating the manufacturing setup rig for sensors (215; 215a, 215b) of drug delivery devices (1) or drug delivery add-on devices is carried out when at least one parameter of the manufacturing setup rig was changed, which has an influence on the determining of the setting of a threshold.

4. The method of claim 1, 2 or 3, wherein the processing of the signal of the reference sensor comprises taking a number of readings of the signal at predefined positions of the reference movable dosage programming component with the reference sensor and the electronic system being supplied with at least one predefined electric voltage supply and within at least one predefined ambient temperature range.

5. The method of claim 4, wherein the processing of the signal of the reference sensor comprises calculating the numbers of taken readings, which are below and which are above a default threshold, and changing the default threshold until the numbers of taken readings, which are below and which are above a default threshold, are nearly equal.

6. The method of claim 4 or 5, wherein the taken readings of the signal of the reference sensor are digitized with an analogue-to-digital converter ADC and the threshold factor TF is calculated as TF=(T−L)/(H−L), wherein T=threshold ADC, L=average of readings <10% maximum, H=average of readings >90% maximum.

7. The method of any preceding claim, wherein the sensor is one of the following: an optical sensing device (215; 215a, 215b); an electromagnetic radiation sensing device; a capacitive sensing device; an inductive sensing device; a magnetic sensing device; an ionising radiation sensing device; an acoustic sensing device; an electric current sensing device; an electric voltage sensing device; an acceleration sensing device; a gyroscopic sensing device.

8. The method of any preceding claim, wherein the movable dosage programming component comprises a rotary encoder system (70a, 70b) having a predefined angular periodicity and during the calibrating and tuning steps a rotation of the rotary encoder system is detected by the sensor.

9. The method of claim 8, wherein the rotary encoder system comprises 12 nearly equally spaced flags (70a, 70b) and the sensor takes 300 samples during a 360° rotation of the rotary encoder system, wherein the sampling times are uniformly distributed over the 360° rotation.

10. The method of any preceding claim, wherein the threshold factor is adjusted for a typical operating voltage of a production drug delivery device or of a production drug delivery add-on device.

11. A manufacturing setup rig for sensors of drug delivery devices or drug delivery add-on devices provided for tuning of at least one threshold of a sensor (215; 215a, 215b) of a drug delivery device (1) or of a drug delivery add-on device, wherein the drug delivery device comprises a movable dosage programming component (70, 70a, 70b) and the sensor (215; 215a, 215b) is provided and configured to detect movement of the movable dosage programming component (70, 70a, 70b) relative to the sensor (215; 215a, 215b) during dosing of a drug by comparing an output signal (1004) of the sensor (215; 215a, 215b) with at least one threshold, and wherein the manufacturing setup rig comprises:
a reference movable dosage programming component;
a reference sensor; and
an electronic system configured for processing a signal generated by the reference sensor upon movement of the reference movable dosage programming component and converting the determined setting into a threshold factor.

12. The manufacturing setup rig of claim 11, wherein at least one of the reference movable dosage programming component, the reference sensor and the electronic system have predefined nominal characteristics being representative of a production drug delivery device or of a production drug delivery add-on device.

13. The manufacturing setup rig of claim 11 or 12, comprising at least one predefined electric voltage supply for the electronic system.

14. The manufacturing setup rig of claim 11, 12 or 13, configured for providing at least one predefined ambient temperature range.

15. A production drug delivery device (1) or of a production drug delivery add-on device, wherein the production drug delivery device (1) comprises a movable dosage programming component (70, 70a, 70b) and the production drug delivery device (1) and/or the production drug delivery add-on device comprise(s) at least one sensor being (215; 215a, 215b) provided and configured to detect movement of the movable dosage programming component (70, 70a, 70b) relative to the at least one sensor (215; 215a, 215b) during dosing of a drug by comparing an output signal (1004) of the at least one sensor (215; 215a, 215b) with at least one threshold, and wherein the at least one threshold of the at least one sensor (215; 215a, 215b) is tuned with a method of any of the claims 1 to 10.
